(19) United States
(12) Patent Application Publication
(10) Pub. No.: US 2015/0105630 A1
(43) Pub. Date: Apr. 16, 2015

(54) HEART PULSE MONITOR INCLUDING A FLUXGATE SENSOR

(71) Applicants: Texas Instruments Incorporated, Dallas, TX (US); TEXAS INSTRUMENTS DEUTSCHLAND GmbH, Freising (DE)

(72) Inventors: STEVEN ALFRED KUMMERL, CARROLLTON, TX (US); ANURAAG MOHAN, FREMONT, CA (US); VIOLA SCHAFFER, FREISING (DE)

(21) Appl. No.: 14/050,910

(22) Filed: Oct. 10, 2013

(51) Int. Cl. A61B 5/0205; A61B 5/00

(52) U.S. Cl. CPC A61B 5/0205; A61B 5/681; A61B 5/4866; A61B 5/742; A61B 5/02444
USPC 600/301; 600/502; 600/503

(57) ABSTRACT

A heart pulse monitor includes a permanent magnet including a mounting structure for securing the permanent magnet in displaceable contact with a blood vessel of a wearer. The permanent magnet has a thickness defining an axial direction that the permanent magnet is displaceable when blood flows. A fluxgate sensor system is positioned a distance in the axial direction from the permanent magnet to sense an axial magnetic field therefrom. The permanent magnet displaces in the axial direction upon a heart pulse of the wearer resulting in a change in the axial magnetic field which is sensed by the fluxgate sensor system through a change in an induced AC output signal on the sense coil. A processor is coupled to receive information from the induced AC output signal. The processor implements calibration data which converts information from the induced AC output signal into a heart pulse measurement for the wearer.

### FIELD

Disclosed embodiments relate to non-invasive heart pulse monitors including magnets.

### BACKGROUND

Conventional 2 point heart pulse measurements require electrodes to be placed across the heart for accurate measurement, typically using a chest strap or electrodes. A strapless contact is also known but requires the user to touch the wrist with the opposite hand in order for the pulse readings to be taken. An alternate known approach uses a reflective pulse oximetry technique, but requires a relatively complex circuit (e.g., a light emitting diode (LED), detector, LED drivers, etc).

### SUMMARY

Disclosed embodiments describe non-invasive heart pulse monitors including a permanent magnet and a mounting structure for securing the permanent magnet in displaceable contact with a blood vessel of a wearer of the heart pulse monitor in combination with a fluxgate sensor system. The permanent magnet has a thickness defining an axial direction in which the permanent magnet becomes displaced when blood flows in the blood vessel. The fluxgate sensor system is positioned a nominal distance in the axial direction from the permanent magnet and is aligned relative to the permanent magnet to sense an axial magnetic field therefrom.

When the permanent magnet displaces in the axial direction due to a heart pulse, a resulting change in the axial magnetic field is sensed by the fluxgate sensor system through a change in an induced alternating current (AC) output signal on its sense coil. A processor is coupled to receive information from the induced AC output signal that applies calibration data which converts the information from the induced AC output signal into a heart pulse measurement for the wearer. Disclosed heart pulse monitors thus enable a simplified single point heart rate/pulse measurement through pulses causing fluctuations in the magnetic field from the permanent magnet sensed by the fluxgate sensor system.

### BRIEF DESCRIPTION OF THE DRAWINGS

Reference will now be made to the accompanying drawings, which are not necessarily drawn to scale, wherein:

FIG. 1A is a depiction of an example heart pulse monitor including a permanent magnet for positioning in displaceable contact with a blood vessel of a wearer of the heart pulse monitor and a fluxgate sensor system, where the fluxgate sensor system senses the movement of the permanent magnet, according to an example embodiment.

FIG. 1B is a depiction of an example fluxgate sensor system embodied as a ring core fluxgate, according to an example embodiment.

FIG. 2 is a depiction of example heart pulse monitor including a permanent magnet and a first and a second fluxgate sensor system, according to an example embodiment.

FIG. 3 is a depiction of an example heart pulse monitor including a permanent magnet and a fluxgate sensor system along with a weight monitoring device that measures caloric output of the wearer, according to an example embodiment.

FIG. 4A depicts the heart pulse monitor configuration including a dual core fluxgate sensor system used for tests described herein.

FIG. 4B depicts the B field magnitude along the fluxgate axis shown for the dual core fluxgate sensor system shown in FIG. 4A.

FIG. 4C shows plots of a simulated B field sensed by the fluxgate sensor system shown in FIG. 4A in mT as a function of the fluxgate height (fgHeight) in mm between the permanent magnet and the fluxgate sensor system, for various fluxgate gaps (fgGaps) between the cores in the dual core fluxgate sensor system.

### DETAILED DESCRIPTION

Example embodiments are described with reference to the drawings, wherein like reference numerals are used to designate similar or equivalent elements. Illustrated ordering of acts or events should not be considered as limiting, as some acts or events may occur in different order and/or concurrently with other acts or events. Furthermore, some illustrated acts or events may not be required to implement a methodology in accordance with this disclosure.

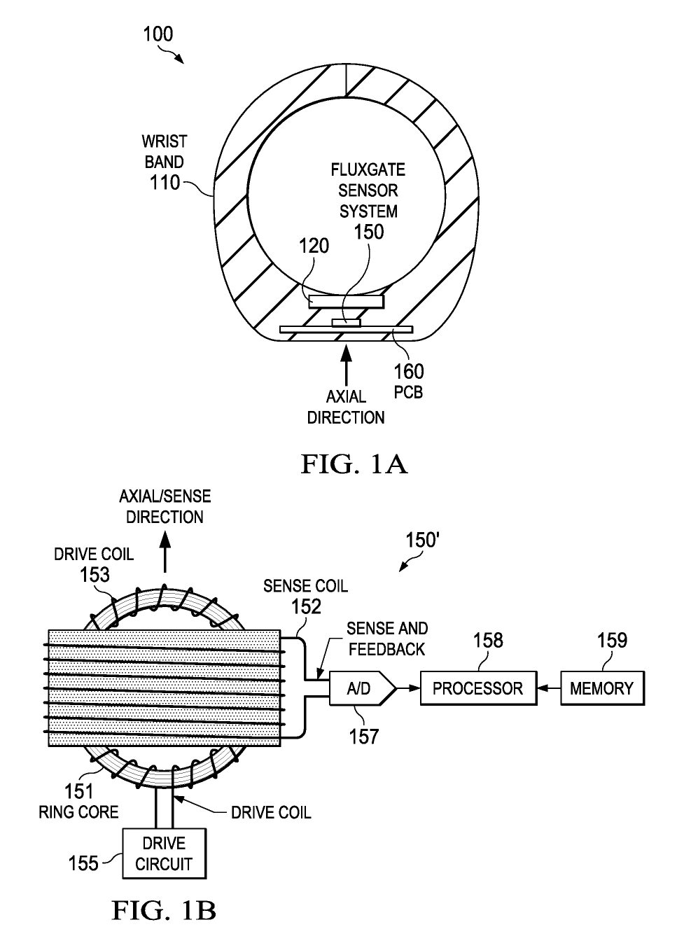

FIG. 1A is a depiction of an example heart pulse monitor 100 including a permanent magnet 120 configured together with a wrist band 110 in displaceable contact with a blood vessel of a wearer of the heart pulse monitor and a fluxgate sensor system 150, where the fluxgate sensor system 150 senses the movement of the permanent magnet 120 in proximity, according to an example embodiment. The proximity is generally on the order of 1 mm, such as 0.02 mm to 30 mm. The earth's magnetic field can set an approximate maximum useful distance between the permanent magnet 120 and the fluxgate sensor system 150.

The fluxgate sensor system 150 is shown mounted on an optional printed circuit board (PCB) 160. The PCB 160 allows for the mounting of electronic circuitry such as IC's (e.g., a low pass filter, amplifier, analog to digital converter (ADC or A/D) and digital signal processor (DSP) for the signal processing of the electrical signal from the sense coil (see sense coil 152 in FIG. 1B described below) of the fluxgate sensor system 150. The respective ICs along with a memory chip for storing algorithms that the processor may implement as described below can all be part of a multi-chip module (MCM).

The heart pulse monitor 100 has a mounting structure shown as the wrist band 110 in the shape of a bracelet that fits on a person's wrist which secures the permanent magnet 120 in “displaceable contact” with respect to a peripheral blood vessel of a wearer of the heart pulse monitor. As used herein, “displaceable contact” refers to a direct or indirect contact so that the permanent magnet 120 experiences a force and becomes displaced in the axial direction when pulses of blood flowing through the blood vessel press the blood vessel adjacent to the permanent magnet 120. The wrist band 110 or other mounting structure can be flexible, which allows the permanent magnet 120 itself to be largely inflexible and still provide axial displacement.

As known in the an of magnets, a permanent magnet is made from a material that is magnetized and creates its own persistent magnetic field (i.e., a ferromagnetic material). Wrist band 110 generally comprises a flexible plastic material having the permanent magnet 120 secured therein. The permanent magnet 120 has a height (or thickness) defining the axial direction shown in which the permanent magnet 120 is displaceable when blood flows in the blood vessel of the wearer.

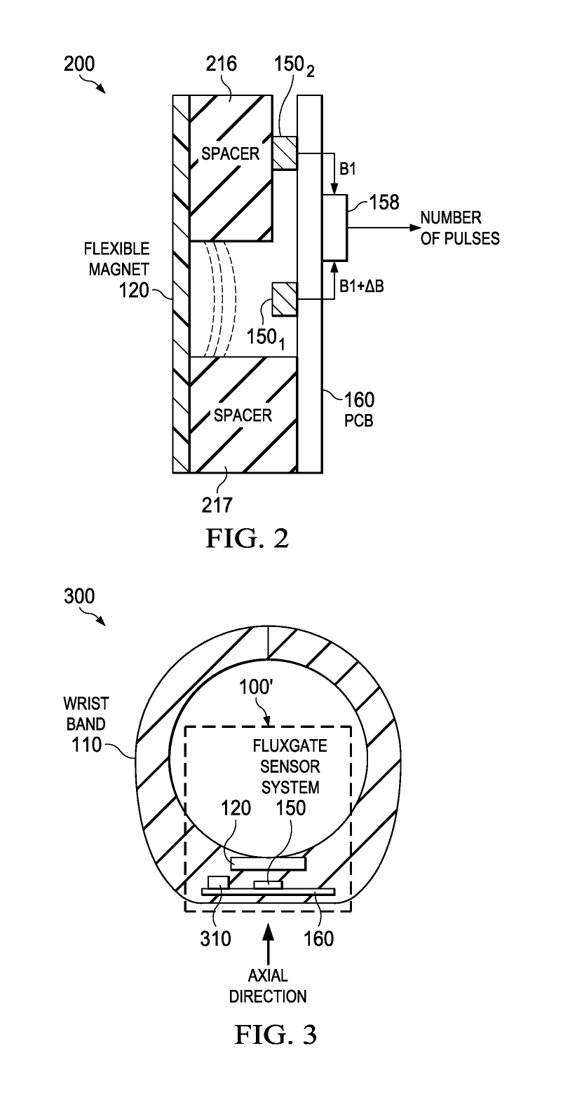

The fluxgate sensor system 150 is positioned at a nominal distance in the axial direction from the permanent magnet 120, such as a nominal distance provided by at least one spacer (see spacers 216 and 217 in FIG. 2 described below). The fluxgate sensor system 150 is aligned relative to the permanent magnet 120 to sense an axial magnetic field of the permanent magnet 120.

The permanent magnet 120 is selected to provide a magnetic field that is primarily in the axial direction shown and provide a suitable magnetic field strength. In one embodiment, the permanent magnet 120 is a poled (programmed) magnet, which can be embodied as a “refrigerator magnet” also known as a Halbach array arrangement. Unlike most conventional magnets that have distinct north and south poles, refrigerator magnets are flat and are made from composite materials (a polymer together with magnetic particles such as nickel (Ni) flakes), which are typically constructed with alternating north and south poles on the same surface of the plane.

Although the mounting structure is shown as a wrist band 110, the mounting structure may be on other locations of the wearer, such as on the upper arm, on the ankle, or on the neck. In each of these body parts, peripheral blood vessels are known to pass through.

FIG. 1B is a depiction of an example fluxgate sensor system 150′ embodied as a ring core fluxgate sensor system', according to an example embodiment. Fluxgate sensor system 150′ includes at least one magnetic core 151 shown as a ring core, and a sense coil 152, and a drive coil 153 both proximate to the magnetic core 151. The name “fluxgate” derives from the action of the magnetic core 151 gating magnetic flux in and out of the sense coil 152. A drive circuit 155 is coupled to drive the drive coil 153. In other embodiments, the fluxgate sensor system can include a first and a second magnetic core.

A processor (e.g., a DSP) 158 is shown coupled to receive a digitized signal having information derived from the induced AC output signal on the sense coil 152, shown including the digitation function provided by an analog-to-digital (A/D) converter 157 coupled to the sense coil 152. Although not shown, a low pass filter and amplifier are also generally included in typical fluxgate sensor systems. The AC output signal on the sense coil 152 is essentially proportional to the magnetic field. Since the magnetic field strength decays (decreases) non-linearly with increasing distance between the permanent magnet 120 and the fluxgate sensor system 150′, non-linearly mathematical processing is generally used to determine the heart pulse of the user. The processor 158 shown in FIG. 1B can implement calibration data stored in the memory 159 shown in FIG. 1B to convert the induced AC output signal into a heart pulse measurement. Calibration data may also be provided by the manufacturer or determined either by simulation or empirically.

As known in the art, the basic principle of operation of a fluxgate sensor system is comparison of a measured magnetic field B, with a reference magnetic field B_{ref}. B_{ref} can have a variety of shapes including sinusoid, square, or a triangle alternating signal. B_{ref} is excited to the magnetic core 151 through the B field from the drive coil 153 while being driven by the drive circuit 155. The magnetic field measured Bₑₓₜ is superposed with B_{ref}. Then Bₑₓₜ is sensed in the magnetic core 151 by the sense coil 152 (pick-up coil) to be evaluated.

The sensitivity of the fluxgate sensor system is dependent on the magnetic core 151 material's magnetic permeability. In sensing operations, when a change occurs in Bₑₓₜ, the induced AC signal output of the sense coil 152 changes. The extent and phase of this change can be analyzed to ascertain the intensity and orientation of the magnetic flux lines. The sense winding signal on the sense coil 152 will be twice the frequency of the drive winding signal on the drive coil 153 because it appears on both its positive and negative half cycles.

The fluxgate outputs on the sense coil 152 are generally rectangular pulses whose frequency varies inversely proportional to the magnetic field. The frequency output of the fluxgate sensor can be converted to voltage using a frequency to voltage converter such as LM2907 (a Frequency to Voltage Converter from National Semiconductor) or an equivalent.

FIG. 2 is a depiction of example heart pulse monitor 200 including a permanent magnet 120 and a first fluxgate sensor system 150₁ and an optional second fluxgate sensor system 150₂, according to an example embodiment. Spacers are shown as 216 and 217 for supporting the permanent magnet 120 on its outside portion and setting the axial distance between the permanent magnet 120 and the first fluxgate sensor system 150₁ and between the permanent magnet 120 and the second fluxgate sensor system 150₂.

The second fluxgate sensor system 150₂ provides an induced AC signal reflecting the B field shown as B1 that after signal processing and digitation through differencing by processor 158 implementing a difference function with the induced AC signal from the first fluxgate sensor system 150₁ shown as B1+ΔB. This arrangement enables tuning out (removing) noise present in the output signal, such in the form AC noise signals induced by proximity to walls of the room, the earth's magnetic field, and other magnetic field distortion sources.

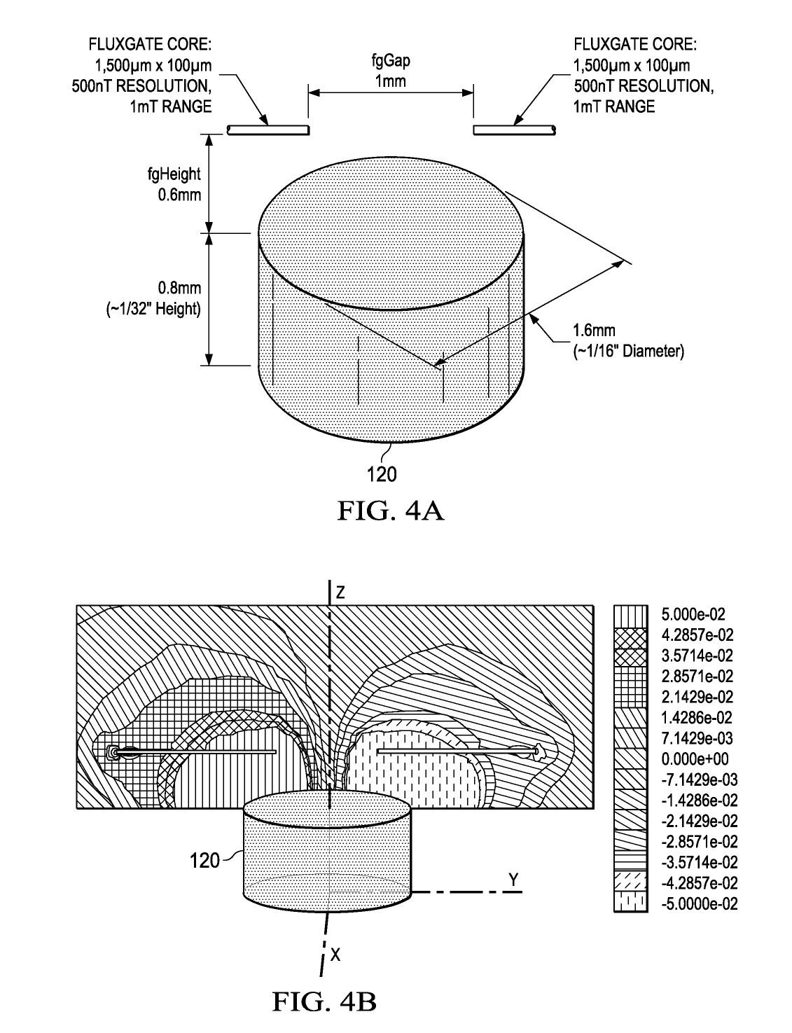

FIG. 3 is a depiction of a health monitoring combination 300 including the sensing portion 100′ of the example heart pulse monitor 100 shown in FIG. 1A including a permanent magnet 120 and a fluxgate sensor system 150 along with a weight monitoring device 310 on the PCB 160 that measures caloric output of the wearer, according to an example embodiment. The weight monitoring device 310 can comprise a heat flux-based device or an accelerometer. Although the weight monitoring device 310 is shown on the same PCB 160 as fluxgate sensor system 150 to facilitate utilizing the same processor as the fluxgate sensor system, the weight monitoring device 310 can be on a separate support structure. Although not shown, the health monitoring combination 300 can also include a monitor or a screen on which the parameters indicating the heart pulse performance and caloric burn rate are displayed for the wearer.

Advantages of disclosed heart pulse monitors having permanent magnets and a fluxgate sensor system include low cost and simple reference design, small size, and enabling a single point non-electrical contact measurement technique. Also, there is no optics needed, so that a line-of-sight is not needed.

### EXAMPLES

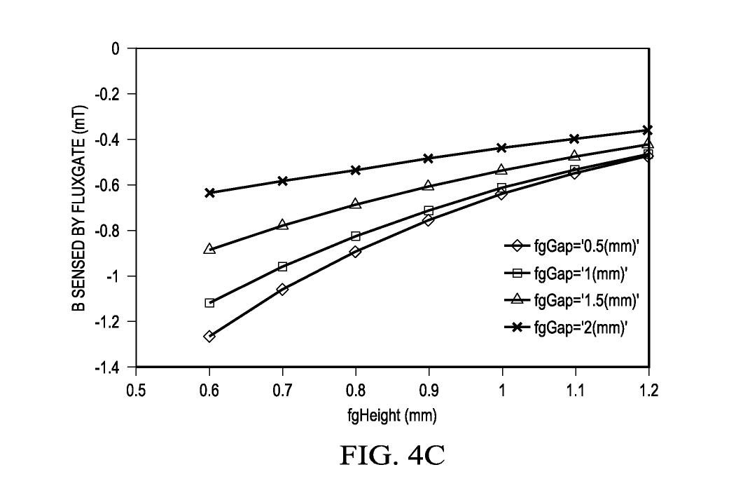

The setup depicted in FIG. 4A was used for field simulations of an example heart pulse monitor having fluxgate sensor system including first and second fluxgate cores 1500 μm×100 μm, with a 500 nT resolution, a 1 mT range, where the gap between the cores (fgGap) was 1 mm. The nominal distance (fgHeight) between the permanent magnet (1.6 mm diameter, 0.8 mm height/thickness) and the in-plane location between the cores shown in FIG. 4A was 0.6 mm. FIG. 4B depicts the B field magnitude along the fluxgate axis (axial direction) shown.

Simulations were repeated for permanent magnets about 50× weaker as compared to NdFeB grade N42, with a surface field strength of about 100 G (10 mT). FIG. 4C shows plots of the simulated B field sensed by the fluxgate sensor system (in mT) as a function of fgHeight (in mm), for various fgGaps. For a FgGap of 1 mm, the field variation with the permanent magnet 120 displaced by pulse is 0.014″ or 356 μm, which corresponds to a 377 μT variation in magnetic field at fluxgate. 005″ or 127 μm corresponds to a 164 μT variation in magnetic field strength at the fluxgate. These field variation magnitudes can be detected by conventional fluxgate sensor systems.

Those skilled in the art to which this disclosure relates will appreciate that many other embodiments and variations of embodiments are possible within the scope of the claimed invention, and further additions, deletions, substitutions and modifications may be made to the described embodiments without departing from the scope of this disclosure.

1. A heart pulse monitor, comprising:
a permanent magnet including a mounting structure for securing said permanent magnet in displaceable contact with a blood vessel of a wearer of said heart pulse monitor, said permanent magnet having a thickness defining an axial direction in which said permanent magnet becomes displaced when blood flows in said blood vessel;
at least a first fluxgate sensor system includes at least one magnetic core, a sense coil and a drive coil proximate to said magnetic core, and a drive circuit coupled to drive said drive coil, wherein said first fluxgate sensor system is positioned at a nominal distance in said axial direction from said permanent magnet and is aligned relative to said permanent magnet to sense an axial magnetic field therefrom, and
wherein when said permanent magnet displaces in said axial direction due to a heart pulse of said wearer, a change results in said axial magnetic field which is sensed by said first fluxgate sensor system through a change in an induced alternating current (AC) output signal on said sense coil,
a processor coupled to receive information from said induced AC output signal, and
wherein said processor implements calibration data which converts said information from said induced AC output signal into a heart pulse measurement for said wearer.

2. The heart pulse monitor of claim 1, wherein said mounting structure comprises a wrist band.

3. The heart pulse monitor of claim 1, wherein said mounting structure is a flexible mount which displaces said permanent magnet in said axial direction responsive to said heart pulse of said wearer.

4. The heart pulse monitor of claim 1, further comprising a second fluxgate sensor system positioned sufficiently laterally away from said first fluxgate sensor system to not sense said change in said axial magnetic field when said permanent magnet displaces in said axial direction.

5. The heart pulse monitor of claim 1, further comprising a weight monitoring device which measures a caloric output of said wearer secured by or positioned within said mounting structure.

6. The heart pulse monitor of claim 1, further comprising a printed circuit board (PCB), wherein said first fluxgate sensor system and said processor are mounted on said PCB.

7. The heart pulse monitor of claim 6, wherein said nominal distance is set by at least one spacer positioned between an outer portion of said permanent magnet and said PCB.

8. The heart pulse monitor of claim 1, wherein said nominal distance is from 0.02 mm to 30 mm.

9. A method of heart pulse monitoring, comprising:
securing a mounting structure of a heart pulse monitor, said heart pulse monitor including a permanent magnet in displaceable contact with a blood vessel of a wearer of said heart pulse monitor, said permanent magnet having a thickness defining an axial direction in which said permanent magnet becomes displaced when blood flows in said blood vessel and at least a first fluxgate sensor system including at least one magnetic core, a sense coil and a drive coil proximate to said magnetic core, and a drive circuit coupled to drive said drive coil, wherein said first fluxgate sensor is positioned a nominal distance in said axial direction from said permanent magnet to sense an axial magnetic field therefrom;
receiving an induced alternating current (AC) output signal from said sense coil responsive to said permanent magnet displacing in said axial direction due to a heart pulse of said wearer resulting in a change of said axial magnetic field which is sensed by said first fluxgate sensor system through a change in said induced AC output signal,
using a processor having calibration data, converting information from said induced AC output signal into a heart pulse measurement for said wearer.

10. The method of claim 9, further comprising displaying said heart pulse measurement.

11. The method of claim 9, further comprising a second fluxgate sensor system positioned sufficiently laterally away from said first fluxgate sensor system to not sense said change in said axial magnetic field when said permanent magnet displaces in said axial direction, and
using information from a background induced AC signal provided by said second fluxgate sensor system, performing a differencing function with said information from said induced AC output signal to reduce background magnetic field distortions influencing said heart pulse measurement.

12. The method of claim 9, wherein a weight monitoring device which measures a caloric output of said wearer is secured by or within said mounting structure, further comprising displaying said caloric output of said wearer.

13. The method of claim 9, further comprising a printed circuit board (PCB), wherein said first fluxgate sensor system and said processor are mounted on said PCB, and wherein said nominal distance is set by at least one spacer positioned between an outer portion of said permanent magnet and said PCB.

14. The method of claim 9, wherein said nominal distance is from 0.02 mm to 30 mm.
